(19) United States
(12) Patent Application Publication
(10) Pub. No.: US 2019/0125694 A1
(43) Pub. Date: May 2, 2019

(54) METHOD FOR TREATMENT OF VISUAL STRESS CONDITIONS AND COMPOSITIONS USED THEREIN

(71) Applicant: OmniActive Health Technologies Limited, Mumbai (IN)

(72) Inventors: Vijaya JUTURU, Morristown, NJ (US); Lynda DOYLE, Morristown, NJ (US); Jayant DESHPANDE, Charlottetown (CA)

(21) Appl. No.: 16/094,017

(22) PCT Filed: Apr. 14, 2017
(86) PCT No.: PCT/IB17/52180

§ 371 Date: Oct. 16, 2018

Related U.S. Application Data
(60) Provisional application No. 62/323,671, filed on Apr. 16, 2016.

(51) Int. Cl. A61K 31/047; A61P 27/02; A61K 9/00

(52) U.S. Cl. CPC A61K 31/047; A61K 9/0053; A61P 27/02

(57) ABSTRACT

The methods relate to preventing photoreceptor damage caused by blue light by oral administration of a macular carotenoid composition comprising of lutein and zeaxanthin isomers. The photoreceptor damage is prevented through the upregulation of genes and protein synthesis manifested by an increase in the levels gene expression and proteins synthesis of Gnat1, Rhodopsin, NCAM, Rod arrestin, GAP-43, Nrf2, Ho-1 and decrease in the GFAP, NFkB (oxidative stress markers) in rat eye models exposed to visible blue light for various time periods and simultaneously treated with macular carotenoid compositions comprising of lutein zeaxanthin and their isomers. Methods described herein also relate to treatment of visual stress conditions by administering macular carotenoid compositions for treatment headache, blurred vision, photoreceptor degeneration, oxidative stress, endoplasmic reticulum stress and the like, which are caused due to prolonged exposure to blue light source. The methods described herein help to treat visual stress disorders by reducing headache and headache frequency, slowing down photoreceptor degeneration, improvement in oxidative stress and protection of retinal cells from light damage. The compositions herein are safe for humans and other animals for consumption and can be used for treatment of visual stress disorders in the subject in need thereof.

### FIELD

Methods herein are described for treatment of photoreceptor damage on exposure to blue light and other visual stress conditions by administering macular carotenoid compositions containing lutein, zeaxanthin and isomers thereof in an effective daily dose, to a subject in need thereof. More particularly the methods relates to prevention of photoreceptor damage on exposure to blue light emitted from digital and electronic devices and light sources and increasing retinal cell viability through upregulation of gene expression and retinal protein synthesis and downregulation of oxidative stress markers by administration of lutein and zeaxanthin composition. Methods are also described for treatment of visual stress conditions such as photoreceptor degeneration and oxidative stress, which are caused due to prolonged exposure to blue light. The compositions used herein are comprised of combination of lutein, meso-zeaxanthin and R,R zeaxanthin, along with one or more food grade excipients such as fat, fatty acid, oil, antioxidant, and vitamin. The methods described herein help to treat visual stress disorders caused by exposure to blue light by slowing down photoreceptor degeneration, improvement in oxidative stress and protection of retinal cells from light damage by upregulation or down regulation of genes.

The compositions herein are safe for humans and other animals for consumption and can be used for treatment of visual stress disorders in the subject in need thereof, when administered ineffective amounts, which can include an effective daily dose.

### BACKGROUND

Exposure to various light sources such as electronic devices, digital devices and sun rays causes stress and gives rise to various eye disorders which affect retinal and brain health. The subjects exposed to light sources of various intensities and wavelengths experience visual stress and recurring headaches which may be caused by overworking the eye muscles in an attempt to better focus their vision. Excessive exposure of eyes to this harmful radiation in the visible spectrum, commonly known as blue light causes photoreceptor damage in the retina. In other cases, problems of internal pressure and swelling within the structures of the eye can ‘refer’ pain into other areas of the head. The cornea and the lens work together to focus images on the retina at the back of the eye. Sometimes, this delicate mechanism fails due to extra stress conditions and overworking and the small muscles of the eye are forced to work harder, causing eye disorders. The result is tired, aching eyes, blurred vision and headache. The eye disease glaucoma can cause severe headaches in some cases. Glaucoma occurs when the drainage area at the back of the eye is blocked, and fluid (aqueous humour) builds up, causing increased pressure (intraocular pressure). The result is damage to the optic nerve. It is not possible to prevent glaucoma from developing, but early detection may help to slow its progression. The slow or rapid build-up of intraocular pressure over time may also cause various eye disorders.

Each day the retina of the average human absorbs approximately 10¹² to 10¹⁵ photons. With greater exposure to light, this hail of photons can cause irreparable damage to the retina. Exposure to light for an extended period of time may result in chemical changes in retinal cells that ultimately result in cell death and is known as photoreceptor damage. The photoreceptor cells involved in vision are the rods and cones. These cells contain a chromophore (11-cis retinal, the aldehyde of Vitamin A1 and light-absorbing portion) bound to cell membrane protein, opsin. Rods deal with low light level and do not mediate color vision. Cones, on the other hand, can code the color of an image. Each cone type responds best to certain wavelengths, or colors, of light because each type has a slightly different opsin. (Hunter et al 2012, ProgRetin Eye Res. 2012 January; 31(1): 28-42)

The rods and cones, which require photo pigments to absorb photons as the first step in seeing, are much more likely to be damaged by excess amounts of visible light. Besides the retinal pigment epithelial cells (RPE) cells contain light absorbers such as melanin, lipofuscin, and retinoids, which make them susceptible to photochemical damage. The study of phototoxicity is all the more important given that eyes are not equally susceptible to light damage. Vulnerability to photochemical damage can depend on many factors including age, diet, and pathology. (Hunter et al 2012, Prog Retin Eye Res. 2012 January; 31(1): 28-42)

Retinal rod cells (also known as photoreceptor cells) are highly differentiated neurons responsible for detecting photons. A specialized part of the rod cell, the rod outer segment (ROS) contains rhodopsin and auxiliary proteins, which convert and amplify the light signal. The system is so exquisitely sensitive that a single photon can be detected. Each mammalian ROS consists of a pancake-like stack of 1000-2000 distinct disks enclosed by the plasma membrane. The main protein component (>90%) of the bilayered disk membranes is light-sensitive rhodopsin. Approximately 50% of the disk membrane area is occupied by rhodopsin, whereas the remaining space is filled with phospholipids and cholesterol. (Palczewski et al Annu Rev Biochem. 2006; 75: 743-767.)

The process by which light is converted into electrical signals in the rod cells, cone cells and photosensitive ganglion cells of the retina of the eye is known as phototransduction. Rod phototransduction is one of the best-characterized G-protein-signaling pathways. The receptor is rhodopsin (R), the G protein is transducin (G), and the effector is cGMP phosphodiesterase (PDE or PDE6). Upon photon absorption, the rhodopsin molecule becomes enzymatically active (R*) and catalyzes the activation of the G protein transducin to G*. Transducin, in turn, activates the effector phosphodiesterase (PDE) to PDE*. PDE* hydrolyzes the diffusible messenger cGMP. The resulting decrease in the cytoplasmic free cGMP concentration leads to the closure of the cGMP-gated channels on the plasma membrane. Channel closure leads to localized reduction on the influx of cations into the outer segment, which results in membrane hyperpolarization, i.e. the intracellular voltage becoming more negative. This hyperpolarization decreases or terminates the dark glutamate release at the synaptic terminal. The signal is further processed by other neurons in the retina before being transmitted to higher centers in the brain and that is how an organism can see things. (Fu et al, University of Utah, Apr. 1, 2010.Webvision: The Organization of the Retina and Visual System [Internet], NCBI bookshelf).

Photoreceptor damage can occur when the energy in a photon of light induces changes in the irradiated molecules, such as changes in electron orbitals, or direct breakage of bonds. For instance, sequential transfer of energy from a photon to a photosensitive molecule, and then to oxygen causes changes in electron orbitals, creating reactive forms of oxygen, such as singlet oxygen. The subsequent reaction of singlet oxygen with surrounding molecules can break their molecular bonds, a process called photoxidation. If too many of these events occur, they can eventually result in cell damage or death. The mechanism of photoreceptor damage depends on the particular type of molecules that act as photosensitizers and the photon energy (related to wavelength) required for inducing a chemical change.(Hunter et al 2012, Prog Retin Eye Res. 2012 January; 31(1): 28-42)

On repeated or long term exposure to damaging light, the retinal cells undergo cell death and proceed further with the phenomenon of retinal remodeling. Retinal remodeling refers to a series of anatomical changes in retinal cells following the loss of relatively large numbers of photoreceptors, occurring over weeks or even months. Three distinct phases have been described from animal models of hereditary retinal degenerations and retinal detachment. Phase 1 events encompass retinal pigment epithelial cells (RPE) stress and photoreceptor degeneration, such as outer segment shortening, misplaced visual pigment, and synapse abnormalities. Phase 2 events include bona fide photoreceptor death, microglial activity, bipolar cell dendrite retraction, and Muller cell hypertrophy including formation of a seal—as distinct from a scar—in the outer retina. Phase 3 involves aberrant neurite formation and synaptogenesis; by this point, remodeling is thought to be irreversible thus “narrowing the window” for therapeutic intervention. (Organisciaket al Prog Retin Eye Res. 2010 March; 29(2): 113-134.)

The rhodopsin gene (Rho) gene encodes for a protein called rhodopsin. Rhodopsin is a light-sensitive receptor protein involved in visual phototransduction. This protein is necessary for normal vision, particularly in low-light conditions. Rhodopsin is found in specialized light receptor cells called rods. As part of the light-sensitive tissue at the back of the eye (the retina), rods provide vision in low light. The rhodopsin protein is attached (bound) to a molecule called 11-cis retinal, which is a form of vitamin A. When light hits this molecule, it activates rhodopsin and sets off a series of chemical reactions that create electrical signals. These signals are transmitted to the brain, where they are interpreted as vision.

(https://ghr.nlm.nih.gov/gene/RHO#resources,Genetics home reference, Lister Hill National Center for Biomedical Communications U.S. National Library of Medicine National Institutes of Health Department of Health & Human Services)

Guanine nucleotide-binding protein G(t) subunit alpha-1 also called as transducin alpha is a protein that in humans is encoded by the GNAT1 gene. Transducinalphais a 3-subunit guanine nucleotide-binding protein (G protein) which stimulates the coupling of rhodopsin and cGMP-phoshodiesterase during visual impulses. The transducin alpha subunits in rods and cones are encoded by separate genes. (https://www.ncbi.nlm.nih.gov/gene/2779)

Rod arrestin gene also known as S-antigen (Sag) gene is responsible to encode for a major soluble photoreceptor retinal protein rod arrestin or arrestin-1 that is involved in desensitization of the photoactivated transduction cascade and is encoded by the rod arrestin gene. It is expressed in the retina and the pineal gland and inhibits coupling of rhodopsin to transducin. Arrestin-1 is the second most abundant protein in rod photoreceptors and is nearly equimolar to rhodopsin. Its well-recognized role is to “arrest” signaling from light-activated, phosphorylated rhodopsin, a prototypical G protein-coupled receptor. (Chen et al, Handb Exp Pharmacol. 2014; 219:85-99. doi: 10.1007/978-3-642-41199-1_4.)

Neural cell adhesion molecule (NCAM) gene is responsible to encode for a cell surface glycoprotein also known as Neural cell adhesion molecule protein, which belongs to the immunoglobulin superfamily of adhesion molecules. The neural cell adhesion molecule (NCAM) is an immunoglobulin-like neuronal surface glycoprotein which binds to a variety of other cell adhesion proteins to mediate adhesion, guidance, and differentiation during neuronal growth.(Wedelji et al Ann Med Surg (Lond). 2014 September; 3(3): 77-81.). NCAM influences cell migration, neurite extension, fasciculation, and formation of synapses in the brain. (Hartz et al ExpGerontol. 1998 November-December; 33(7-8):853-64.). Polysialic acid (PSA) is a carbohydrate attached to NCAM via either of two specific sialyltransferases: ST8SiaII and ST8SiaIV. Polysialylated neural cell adhesion molecule (PSA-NCAM) mediates cell interactions, plays a role in axon growth, migration, synaptic plasticity during development and cell regeneration. Some evidence has shown that PSA-NCAM supports the survival of neurons after any kind of injury like ischemia, photo damage etc. It was demonstrated that PSA-NCAM is present in abundance in the retina during development and in adulthood. Neural cell adhesion molecule (NCAM) plays an important role in supporting the survival of injured retinal ganglion cells. (Lobanovskaya et al, Brain Res. 2015 Nov. 2; 1625:9-17. doi: 10.1016/j.brainres.2015.08.008. Epub 2015 Aug. 28.)

Growth associated protein 43 gene is responsible to encode for Growth associated protein 43 (GAP-43).Growth associated protein 43 also known as B-50 or neuromodulin, is a membrane-associated protein that is highly expressed in neuronal growth cones during synaptogenesis in the CNS and is downregulated after synaptogenesis in most brain regions except in specific brain regions that retain plasticity. In the adult retina GAP-43 is localized in the inner plexiform layer as a result of expression by ganglion cells and a subset of amacrine cells. Following retinal injury or optic nerve transection, GAP-43 expression is transiently increased This suggests a structural remodeling in the inner plexiform layer following injury in order to preserve retinal function (Isenmann and Bahr 1997, Isenmann et al., 2003). In response to injury, the adult mammalian retina shows signs of structural remodeling, possibly in an attempt to preserve or regain some of its functional neural connections. GAP-43 is a marker for neuronal remodeling and is involved in synapse formation. (Dijk et al, Molecular Ophthalmogenetics, Netherlands Institute for Neuroscience, Experimental Eye Research 84(5):858-67, June 2007)

Glial fibrillary acidic protein (GFAP) is expressed in large quantities in retinal Muller cells and involves injury-dependent signaling. The retina also responds similarly to “stress” but the up-regulation of intermediate filaments occurs primarily in the Muller cells, the radial glia of the retina. This is a remarkably ubiquitous response in that a similar up-regulation can be observed in numerous forms of retinal degeneration. As a consequence of retinal detachment, “mechanical” or photoreceptor injuries to the retina, GFAP, dramatically increase in Muller cells. (Lewis et al, International Review of Cytology 230:263-90⋅February 2003)

Exposure of retinas to damaging blue LED light induces oxidative stress causing the formation of reactive oxygen species and S-opsin aggregation. The rapid reactive oxygen species increase leads to mitochondrial damage and activation of the MAP kinase (MAPK) pathway or the nuclear translocation of NF-κB. Activated MAPK and NF-κB induces the activation of caspase and leads to apoptotic retinal cell death. Active NF-κB also activates autophagy, and excessive autophagy leads to retinal cell death. While, S-opsin aggregation causes endoplasmic reticulum (ER) stress. Blue LED light-induced retinal photoreceptor-derived cell death is thus associated with both oxidative stress and ER stress. (Kuse et al Sci Rep. 2014; 4: 5223.)

The retinal cells have highly developed endogenous antioxidant defense systems to counteract the oxidative stress generated on exposure to excessive light. Antioxidant/electrophile response element (ARE/EpRE)-regulated phase II detoxifying enzymes and antioxidants is one of the major antioxidant pathways involved in counteracting increased oxidative stress and maintaining the redox status in many tissues. Heme oxygenase-1 (HO-1), the rate-limiting enzyme that catalyzes the degradation of heme to biliverdin, carbon oxide (CO) and iron, is one of the ARE-regulated phase II detoxifying enzymes and antioxidants, which are regulated by the redox-sensitive transcription factor nuclear factor erythroid 2-related factor (Nrf2). Nrf2 demonstrates a protective role against photo-oxidative or chemical stress, the activated Nrf2 translocates into the nucleus, and interacts with ARE, then antioxidant genes are activated to attenuate cellular oxidative stress. (He et al, PLoS One. 2014; 9(1): e84800.Published online 2014 Jan. 6.) The expression of photoreceptor genes like rhodopsin gene (Rho) gene, GNAT-1 gene, Rod arrestin gene also known as S-antigen (Sag) gene, NCAM gene, Growth associated protein 43 (GAP-43)genes are downregulated due to apoptosis and cell death on exposure to damaging blue light. Photoreceptor damage also downregulates certain proteins like nuclear transcription factor Nrf2, HaemeoxygenaseHO-1 which are responsible for photoinjury related retinal repair mechanisms. Also the retinal oxidative stress markers such as NFkB, GFAP protein are upregulated in response to oxidative stress and cell death on photoreceptor damage. These changes adversely impact the process of photo transduction and cause vision loss.

The optic nerve transmits information from the eye to the brain. Brain tumours, haemorrhages or swelling are just some of the disorders that can cause the optic nerve to swell with excess fluid. This also gives rise to headaches and eye stress condition without showing major symptoms. Extended computer use or inadequate or excessive lighting may also cause various eye disorders, thus affecting retinal health, although there are no permanent consequences of this light exposure. Due to intense and continuous use of eyes such as driving a car for extended periods, reading, or working at the computer, the person may feel ocular fatigue, tired eyes, blurring of vision, frequent headaches and occasionally doubling of the vision. Visually intense task such as reading fine prints, using the computer for hours at a time, or trying to see in the dark, unconsciously clench the muscles of eyelids, face, temples, and jaws and develop discomfort or pain. Common precipitating factors for the onset of eye stress disorders include extended use of a computer or video monitor, straining to see in very dim light, and exposure to extreme brightness or glare. Many people will blink less than normal when performing extended visual tasks. This decreased blinking may lead to dryness of the ocular surface and symptoms of dry eyes. Refractive errors (a need for glasses for distance or near vision, or both) may produce the symptoms of eye stress and cause headache.

The refractive errors and eye conditions may result in eyestrain headaches arepresbyopia, hyperopia, and astigmatism. In addition to this, oxidative stress is another factor contributing to eye stress disorders causing headaches and lead to morphological changes in eye. Oxidative stress and endoplasmic reticulum (ER) stress are major factors underlying photoreceptor degeneration in retinitis pigmentosa (RP).

US20070082066 discloses a method for reducing light hyper-sensitivity and various related conditions (such as migraine or other recurrent headaches) by administering dietary carotenoid i.e. zeaxanthin, wherein the preferred dosage ranges from about 10 to about 100 mg per day. The zeaxanthin can also be administered along with additional active agent selected from the group consisting of lutein, extracts from bilberries, wolfberries, gingko biloba, or green tea, Vitamins C or E, zinc, and plant flavonoids, isoflavones, polyphenols, and anthocyanins.

Patent application US 20130231297discloses an orally administered composition for improving visual performance, particularly to reduce eye fatigue or enhance at least one of visual acuity, contrast acuity, glare relief and recovery, and high intensity blue light filtration; wherein the said composition comprising astaxanthin in an amount of about 2 mg to about 12 mg; saffron in an amount of about 5 mg to about 30 mg; lutein in an amount of about 2 mg to about 16 mg; zeaxanthin in an amount of about 0.5 mg to about 5 mg; and European black currant extract in an amount of about 30 mg to about 130 mg. Further the said composition is also useful to reduce symptoms of eye fatigue such as sensitivity to glare, headaches, sore eyes and blurred vision resulting from extended use of visual display terminals.

U.S. Pat. No. 6,955,430 relates to externally-worn eye wear preferably sunglasses which comprises a wavelength transmission blocker having a lutein assimilated therein for preventing free radical damage in the eye, the wavelength transmission blocker being sized and configured to maximize visual acuity and blockage of transmission of light wavelengths throughout a blue light spectrum set in a range from about 400 nanometers to about 510 nanometers.

### SUMMARY

The above references do not discuss the effect of macular carotenoids alone in the method of preventing photoreceptor damage caused by blue light coming from digital devices, electronic devices and other light sources such as LEDoron treatment of eye disorders such as headache, oxidative stress and photoreceptor degeneration caused due to exposure of eyes to light sources having different intensities and thus improving retinal and brain health. The references do not relate to the evaluation of macular carotenoids or their compositions for their effect on recovery of visual stress disorders such as headache in a subject in need thereof. Applicant has carried out rigorous experimentation for use of macular carotenoids compositions and evaluation of the results ef which demonstrate the effects on treating eye stress disorders such as headache and improvement in oxidative stress and endoplasmic reticulum stress, in a subject who is exposed to electronic devices, digital devices and other light sources such study has not been reported in the references.

Administration of macular carotenoid composition comprising lutein and zeaxanthin isomers helps in preventing photoreceptor damage caused by blue light through upregulation of photoreceptor genes and downregulation of markers of oxidative stress. More particularly, the methods relates to method of treating photoreceptor damage caused by exposure to blue light by upregulation of retinal photoreceptor gene NCAM responsible for neurite growth and upregulation of GNAT1 photoreceptor gene responsible for biosynthesis of alpha-transducin 1. The inventors have also found that administration of macular carotenoid composition upregulates the other retinal genes encoding for proteins involved in phototransduction and down regulates the markers of oxidative stress.

In an embodiment, a method herein comprises identifying a subject in need of treatment from eye stress disorder, and administering a composition comprising macular carotenoids alone or in combination with other nutrients, and one or more food grade excipients. The method described herein helps to treat eye stress disorder such as headache, oxidative stress, photoreceptor degeneration, and endoplasmic reticulum stress. The composition also improves retinal and brain health, thus relieving eye stress caused due to prolonged exposure to light sources. The method described herein treats the eye disorders caused due to stress in a subject, who is exposed to light of varying wavelength and light intensities for a prolonged time period. The composition used therein is safe for human and other animals for consumption and can be used for protection and treatment of visual fatigue thereof, when administered in an effective amount which may include an effective daily dose.

In some embodiments, methods of treatment herein are directed to administering a macular carotenoid composition in an effective amount(s) to treat eye stress disorders and photoreceptor damage in a subject caused by prolonged exposure to blue light sources such as the digital devices like television sets, computers, laptops, smart phones and tablets, electronic devices, and fluorescent and LED lighting.

In some embodiments, methods of treatment herein are directed to administering a macular carotenoid composition in an effective amount to a subject in need thereof, and evaluating parameters such as headache frequency, eye fatigue, retinal damage and degeneration, retinal function, oxidative stress and endoplasmic reticulum stress to check the beneficial effects of macular carotenoids for treatment of various eye stress disorders caused by exposure to light sources for prolonged time period.

In some embodiments, compositions and methods of treatment herein are directed to evaluating the effect(s) of a macular carotenoid composition on the treatment of eye stress disorders by administering to a subject in need thereof, an effective amount of a composition comprising macular carotenoid(s) alone or in combination with other nutrients. The macular carotenoid(s) of the composition may be a combination of lutein and zeaxanthin isomers, such as meso-zeaxanthin and R,R zeaxanthin.

In some embodiments, macular carotenoid compositions herein and methods of treatment using the compositions are directed to the treatment of eye disorders caused due to light stress by treating photoreceptor damage, thus relieving the stress and/or discomfort. The methods described herein treat the eye disorders in subject in need thereof such treatment.

In an embodiment, a macular carotenoid composition herein is administered in a daily dose of at or about 0.1 to 100 mg/kg body weight.

In an embodiment, a macular carotenoid composition herein is administered in a daily dose of at or about 1 to 100 mg/kg body weight.

In some embodiments, macular carotenoid compositions herein and methods of treatment using the composition are directed to administering the composition, for example in a daily dose of about 1 mg to about 80 mg/kg body weight of macular carotenoids, to the subject in need thereof.

Further the composition may be administered such that, macular carotenoids contained therein are provided in a daily dose of about 5 mg-50 mg/kg body weight of macular carotenoid composition over a certain amount of treatment to treat visual fatigue.

Further the composition may be administered such that, macular carotenoids contained therein are provided in a daily dose of about 10 mg to 60 mg/kg body weight.

In an embodiment, a method as described herein is directed to administering a carotenoid composition in a daily dose of macular carotenoids such that the combination of 5 mg of lutein and 1 mg of zeaxanthin isomers comprising meso-zeaxanthin and R,R zeaxanthin are provided to a subject in need thereof.

In an embodiment, a method as described herein is directed to administering a carotenoid composition in a daily dose of macular carotenoids such that combination of 7.5 mg lutein and 1.5 mg zeaxanthin isomers is provided to subjects in need thereof, suffering from eye stress disorders. The zeaxanthin isomers comprise meso-zeaxanthin and R,R zeaxanthin.

In an embodiment, a method as described herein is directed to administering a carotenoid composition in a daily dose of macular carotenoids such that combination of 10 mg lutein and 2 mg zeaxanthin isomers is provided to subjects in need thereof, suffering from eye stress disorders.

In an embodiment, a method as described herein is directed to administering carotenoid composition in a daily dose of a combination of 20 mg lutein and 4 mg zeaxanthin isomers is provided to subjects in need thereof, for treatment of eye stress disorders.

In an embodiment, a test composition can be scaled to the doses described above. For example, about 0.1 mg/kg body weight of test composition (e.g. based on the amount of lutein) can include 0.1 mg of lutein and 0.02 mg of zeaxanthin isomers. In another example, about 1 mg of test composition can include 1 mg lutein and 0.2 mg zeaxanthin isomers. In another example, 50 mg of test composition can include 50 mg lutein and 10 mg zeaxanthin isomers. In another example and 100 mg test composition can include 100 mg lutein and 20 mg zeaxanthin isomers. It will be appreciated that in an embodiment, the ratio of lutein and zeaxanthin isomers is at or about 5:1 in the test composition. Accordingly about ⅕^{th} the amount of zeaxanthin isomer content is present in the composition with an amount of lutein.

In one aspect the methods relate to preventing photoreceptor damage caused by blue light by oral administration of a carotenoid composition comprising of lutein and zeaxanthin isomers through up-regulation of GNAT1 which is a photoreceptor gene responsible for biosynthesis of alpha-transducin 1.

In another aspect the methods relate to preventing photoreceptor damage caused by blue light by oral administration of carotenoid composition comprising of lutein and zeaxanthin isomers through up-regulation of NCAM, which is a gene responsible for the neurite growth.

In one more aspect the methods relates to preventing photoreceptor damage caused by blue light by oral administration of carotenoid composition comprising combination of lutein and zeaxanthin isomers, which cause reduction in the level of oxidative stress markers such as GFAP, NFkB.

In one more aspect the methods relate to preventing photoreceptor damage caused by blue light by oral administration of carotenoid composition comprising of lutein and zeaxanthin isomers through upregulation of protein nuclear transcription factor Nrf2, Haemeoxygenase HO-1.

In yet another aspect the methods relate to preventing photoreceptor damage caused by blue light by oral administration of carotenoid composition comprising of lutein and zeaxanthin isomers through upregulation of rhodopsin (Rho) gene, Rod arrestin gene (Sag) gene, Growth associated protein 43 (GAP-43) genes.

In another aspect the methods relate to preventing photoreceptor damage caused by blue light wherein the GNAT1 and NCAM genes are upregulated about 1.2 to 4.5 times in subjects administered with macular carotenoid composition as compared to subjects on vehicle control group.

In one more aspect the methods relate to preventing photoreceptor damage caused by blue light wherein the blue light is coming from digital devices, electronic devices, fluorescent light and LED sources.

The methods described herein relate to treatment of eye stress disorders and prevention of photoreceptor damage caused by blue light in subjects in need thereof, such as for example mammals including a human, who are exposed to blue light for a prolonged time period, by administering an effective amount of macular carotenoid composition and evaluating one or more parameters such as effect on headache recovery, effect on oxidative stress, effect on photoreceptor degeneration and effect on endoplasmic reticulum stress, to check beneficial effects of macular carotenoids for treatment of subjects with various eye stress disorders.

### DETAILED DESCRIPTION OF FIGURES

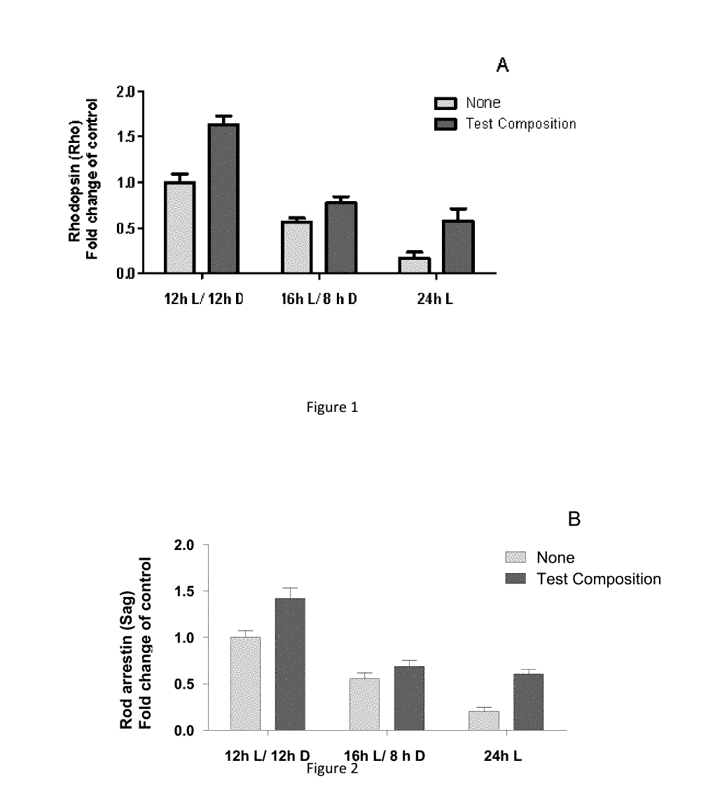

FIG. 1 relates to the graphical representation of the rhodopsin gene expression measured in rat eyes in the treatment and vehicle control groups in terms of fold change of vehicle control group exposed to different light conditions i.e. 24 hour LED light exposure (24 h L), 16 hour light and 6 hour dark adaptation (16 h L/6 h D) and 12 hour light and 12 hour dark adaptation (12 h L/12 h D).

FIG. 2 relates to the graphical representation of the rod arrestin (Sag) gene concentration measured in rat eyes in the treatment and vehicle control groups in terms of fold change of vehicle control group exposed to different light conditions i.e. 24 hour LED light exposure (24 h L), 16 hour light and 6 hour dark adaptation (16 h L/6 h D) and 12 hour light and 12 hour dark adaptation (12 h L/12 h D).

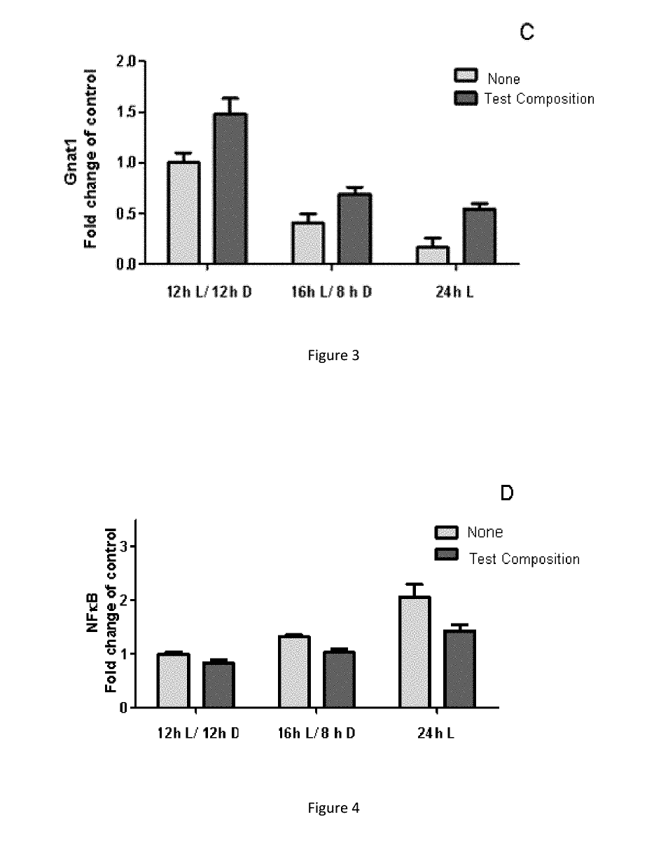

FIG. 3 relates to the graphical representation of the graphical representation of the G protein subunit alpha transducin 1 (GNAT-1)gene expression measured in rat eyes in the treatment and vehicle control groups in terms of fold change of vehicle control group exposed to different light conditions i.e. 24 hour LED light exposure (24 h L), 16 hour light and 6 hour dark adaptation (16 h L/6 h D) and 12 hour light and 12 hour dark adaptation (12 h L/12 h D).

FIG. 4 relates to the graphical representation of the graphical representation of the nuclear factor-kappa B (NF-κB) protein concentration measured in rat eyes in the treatment and vehicle control groups in terms of fold change of vehicle control group exposed to different light condition i.e. 24 hour LED light exposure (24 h L), 16 hour light and 6 hour dark adaptation (16 h L/6 h D) and 12 hour light and 12 hour dark adaptation (12 h L/12 h D).

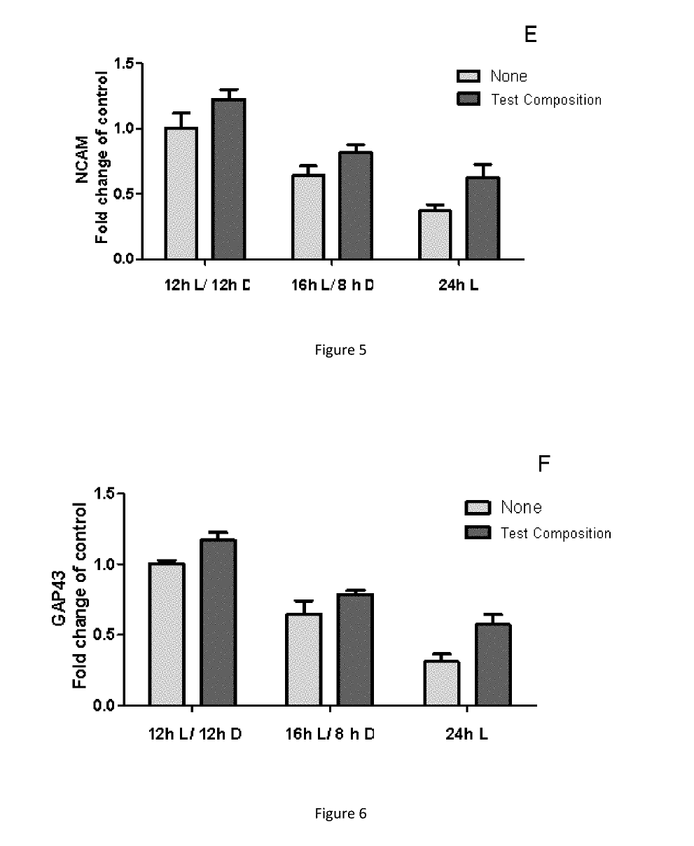

FIG. 5 relates to the graphical representation of the graphical representation of neural cell adhesion molecule (NCAM) gene expression measured in rat eyes in the treatment and vehicle control groups in terms of fold change of vehicle control group exposed to different light conditions i.e. 24 hour LED light exposure (24 h L), 16 hour light and 6 hour dark adaptation (16 h L/6 h D) and 12 hour light and 12 hour dark adaptation (12 h L/12 h D).

FIG. 6 relates to the graphical representation of the graphical representation of the growth-associated protein-43 (GAP-43) gene expression measured in rat eyes in the treatment and vehicle control groups in terms of fold change of vehicle control group exposed to different light conditions i.e. 24 hour LED light exposure (24 h L), 16 hour light and 6 hour dark adaptation (16 h L/6 h D)and 12 hour light and 12 hour dark adaptation (12 h L/12 h D).

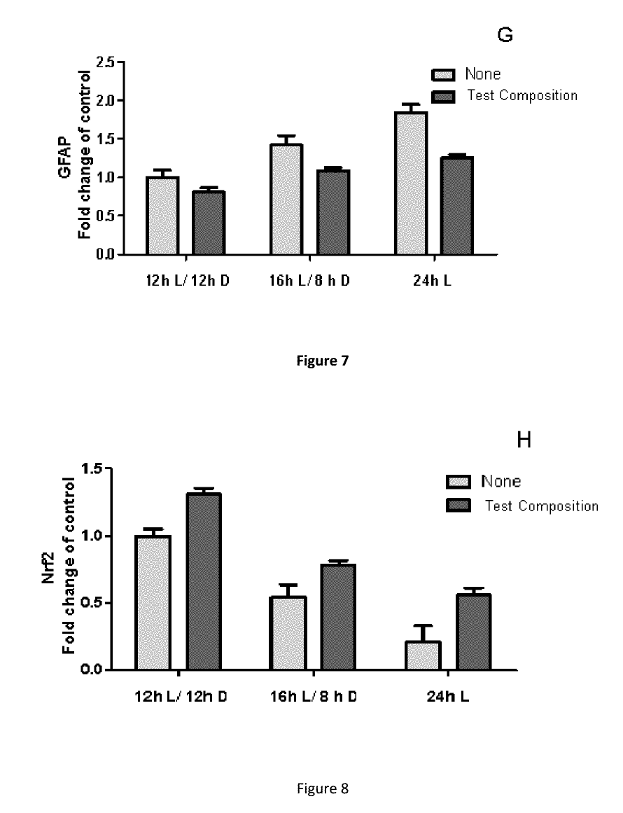

FIG. 7 relates to the graphical representation of the glial fibrillary acidic protein (GFAP) protein concentration measured in rat eyes in the treatment and vehicle control groups in terms of fold change of vehicle control group exposed to different light conditions i.e. 24 hour LED light exposure (24 h L), 16 hour light and 6 hour dark adaptation (16 h L/6 h D) and 12 hour light and 12 hour dark adaptation (12 h L/12 h D).

FIG. 8 relates to the graphical representation of the nuclear factor (erythroid-derived 2)-like 2 (Nrf2) protein concentration measured in rat eyes in the treatment and vehicle control groups in terms of fold change of vehicle control group exposed to different light conditions i.e. 24 hour LED light exposure (24 h L), 16 hour light and 6 hour dark adaptation (16 h L/6 h D) and 12 hour light and 12 hour dark adaptation (12 h L/12 h D).

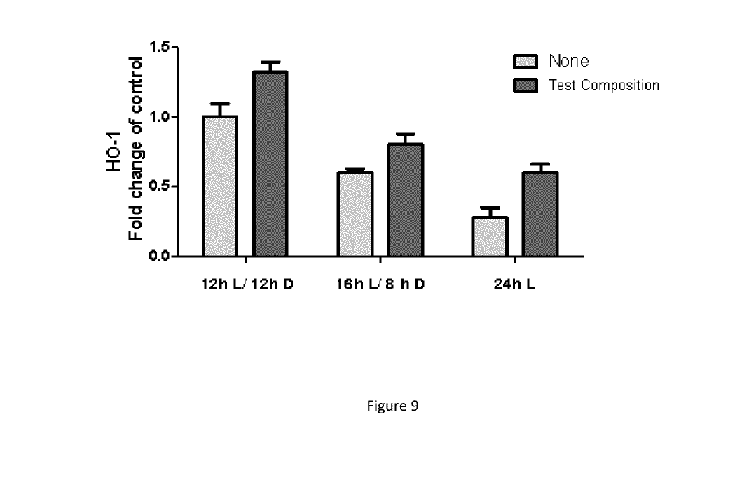

FIG. 9 relates to the graphical representation of the graphical representation of the Haemeoxygenase I (HO-1) protein concentration measured in rat eyes in the treatment and vehicle control groups in terms of fold change of vehicle control group exposed to different light conditions i.e. 24 hour LED light exposure (24 h L), 16 hour light and 6 hour dark adaptation (16 h L/6 h D) and 12 hour light and 12 hour dark adaptation (12 h L/12 h D).

### DETAILED DESCRIPTION

Retinal photoreceptor cells are uniquely adapted to function over a wide range of ambient light conditions. However, in most species prolonged intense visible light exposure can lead to photoreceptor cell damage. In nocturnal animals, the light intensity required for visual cell damage need be only 2-3 times above normal room lighting (Noell 1965). Visual cell damage can then progress to cell death and loss of vision, or “retinal light damage” may regress with recovery of function. Intense light exposure has been used as an environmental stressor in testing genetic animal models of photoreceptor cell degeneration. There is growing evidence that disease mechanisms in age related macular degeneration (AMD) involve oxidative stress and inflammation. Hence it becomes important to understand eye stress disorders caused due to prolonged exposure to light sources, to understand effect on retinal and brain health. As the disorders caused are not reversible, the damaging effects on eye health need to be identified in time and corrective measures need to be taken to avoid further damage to eye.

The methods described herein and the compositions used are directed to treating eye stress disorders by administering a macular carotenoid composition in an effective amount(s) to a subject in need thereof. The methods and compositions herein can treat conditions associated with eye stress, so as to protect and treat eye and improve coordination with brain, when administered for example to a subject who is exposed to various light emitting devices, including but not limited to blue light, sun rays, and other lightwaves of various intensities for a prolonged time period.

The active ingredients of macular carotenoid compositions herein are obtained by natural resources and the compositions overall are safe for administration, and thus the compositions are useful as nutraceutical compositions and/or formulations.

Constant exposure to blue light poses hazard through a modern life style and work conditions and may cause suffering of visual health to a great extent. Such people experience symptoms such as frequent and intense headache, affected vision due to photoreceptor degeneration and oxidative stress and visual stress which may have an overall impact on work performance. One of the causative factors is use of computers and laptops and exposure to blue light for extended periods of time such as for example, but not limited to 4 to 10 hours.

The term blue light used herein refers to the light in the visible spectrum in the range of 400 to 490 nm which is emitted from displays of digital devices such as computers, television laptops, smart phones and tablets, electronic devices fluorescent and LED lighting etc.

The term ‘fold change of the vehicle control group’ means representation of values of the treatment groups in terms of multiples of the values for the vehicle control group exposed to the same LED blue light conditions.

The terminology “subject” refers to a human individual or a mammal which may be undergoing testing, which is being treated with the macular carotenoid compositions herein.

The terminology “subject in need thereof” can include specific individuals or mammals, who may get exposed to, or may have been exposed to blue light for a prolonged period of time such as for example but not limited to 4 to 10 hours. This causes damage to retinal and brain health and damages the photoreceptors and affects retinal cell viability.

The terminology “eye stress disorders” can include various eye disorders caused due to prolonged exposure to light sources, the disorders include intense and frequent headache, affected vision due to photoreceptor degeneration, increased oxidative stress, thus affecting overall retinal and brain health.

In one aspect the methods relate to preventing photoreceptor damage caused by blue light by oral administration of a carotenoid composition comprising of lutein and zeaxanthin isomers through up-regulation of GNAT1 which is a photoreceptor gene responsible for biosynthesis of alpha-transducin 1. The method of preventing photoreceptor damage caused by blue light is obtained by administration of an effective amount of multicarotenoid composition comprising lutein, zeaxanthin or isomers thereof in the dose range of 0.1-100 mg/kg body weight in an embodiment. In an embodiment, the dose range is 1-100 mg/kg body weight, more preferably in the range of 10-80 mg/kg body weight and most preferably in the range of 20-50 mg/kg body weight. In one embodiment the effective amount of the macular carotenoid composition comprises of lutein, meso-zeaxanthin and R,R zeaxanthin in the range from 0.1 mg to 100 mg/kg body weight. Further lutein and zeaxanthin isomers may be present in the ratio of 5:1 in such dose.

In another aspect the methods relate to preventing photoreceptor damage caused by blue light coming by oral administration of an effective amount of carotenoid composition comprising of lutein and zeaxanthin isomers through up-regulation of NCAM gene responsible for the neurite growth. The method of preventing photoreceptor damage caused by blue light is obtained by administration of a multicarotenoid composition comprising lutein, zeaxanthin or isomers thereof in the dose range of 0.1-100 mg/kg body weight in an embodiment. In an embodiment, the dose range is 1-100 mg/kg body weight, more preferably in the range of 10-80 mg/kg body weight and most preferably in the range of 20-50 mg/kg body weight. In one embodiment the effective amount of the macular carotenoid composition comprises of lutein, meso-zeaxanthin and R,R zeaxanthin in the range from 0.1 mg to 100 mg/kg body weight. Further lutein and zeaxanthin isomers may be present in the ratio of 5:1 in such dose.

In one more aspect the methods relate to preventing photoreceptor damage caused by blue light by oral administration of carotenoid composition comprising of lutein and zeaxanthin isomers reduction in the level of oxidative stress markers such as GFAP, NFkB. The method of preventing photoreceptor damage is obtained by administration of a multicarotenoid composition comprising lutein, zeaxanthin or isomers thereof in the dose range of 0.1-100 mg/kg body weight in an embodiment. In an embodiment, the dose range is 1-100 mg/kg body weight, more preferably in the range of 10-80 mg/kg body weight and most preferably in the range of 20-50 mg/kg body weight. In one embodiment the effective amount of the macular carotenoid composition comprises of lutein, meso-zeaxanthin and R,R zeaxanthin in the range from 0.1 mg to 100 mg/kg body weight. Further lutein and zeaxanthin isomers may be present in the ratio of 5:1 in such dose.

In one more aspect the methods relate to preventing photoreceptor damage caused by blue light by oral administration of carotenoid composition comprising of lutein and zeaxanthin isomers through upregulation of protein nuclear transcription factor Nrf2, Haemeoxygenase HO-1.The method of preventing photoreceptor damage is obtained by administration of administration of an effective amount of a multicarotenoid composition comprising lutein, zeaxanthin or isomers thereof in the dose range of 0.1-100 mg/kg body weight in an embodiment. In an embodiment, the dose range is 1-100 mg/kg body weight, more preferably in the range of 10-80 mg/kg body weight and most preferably in the range of 20-50 mg/kg body weight. In one embodiment the effective amount of the macular carotenoid composition comprises of lutein, meso-zeaxanthin and R,R zeaxanthin in the range from 0.1 mg to 100 mg/kg body weight. Further lutein and zeaxanthin isomers may be present in the ratio of 5:1 in such dose.

In yet another aspect the methods relate to preventing photoreceptor damage caused by blue light by oral administration of carotenoid composition comprising of lutein and zeaxanthin isomers through upregulation of rhodopsin (Rho) gene, Rod arrestin gene (Sag) gene, Growth associated protein 43 (GAP-43) genes. The method of preventing photoreceptor damage is obtained by administration of an effective amount of multicarotenoid composition comprising lutein, zeaxanthin or isomers thereof in the dose range 0.1-100 mg/kg body weight in an embodiment. In an embodiment, the dose range is 1-100 mg/kg body weight, more preferably in the range of 10-80 mg/kg body weight and most preferably in the range of 20-50 mg/kg body weight. In one embodiment the effective amount of the macular carotenoid composition comprises of lutein, meso-zeaxanthin and R,R zeaxanthin in the range from 0.1 mg to 100 mg/kg body weight. Further lutein and zeaxanthin isomers may be present in the ratio of 5:1 in such dose.

The method of preventing photoreceptor damage caused by blue light by oral administration of a macular carotenoid composition gives a multi fold increase in the expression of photoreceptor genes as compared to the control vehicle group in groups exposed to different duration of LED blue light. The treatment group treated with the multicarotenoid composition results in upregulation of 1.2 to 4.5 fold increase in the expression of photoreceptor genes, retinal proteins as compared to subjects on the vehicle control group. The methods herein result in the decrease in the expression of oxidative stress markers. The oxidative stress markers show a 0.5 to 3 fold decrease in the treatment group as compared to the vehicle control group. The advantageous effect for the group treated with the multicarotenoid composition is quantified in terms of increase in the expression of the retinal photoreceptor gene expression and protein synthesis as multiple of the value obtained for the vehicle control group and decrease in the expression of the oxidative stress markers as multiples of the value obtained for the vehicle control group.

In another aspect methods of preventing photoreceptor damage caused by blue light involve upregulation of GNAT 1, a photoreceptor gene responsible for biosynthesis of alpha-transducin 1, by about 1.2 to 4.5 times in subjects administered with macular carotenoid composition as compared to subjects on the vehicle control group.

In another aspect method of preventing photoreceptor damage caused by blue light involve upregulation of NCAM a gene responsible for neurite growth by about 1.2 to 4.5 times in subjects administered with macular carotenoid composition as compared to subjects on the vehicle control group.

In an embodiment, methods are described herein for treatment of eye stress disorders by administering a macular carotenoid composition to a subject in need thereof, and evaluating one or more parameters, such as for example but not limited to, improvement in intense and frequent headache, improvement in vision due to improvement in photoreceptor degeneration, reduction in oxidative and endoplasmic reticulum stress to assess improvement in overall retinal and brain health.

In one embodiment, macular carotenoid compositions herein and methods of treatment herein may include administering to a subject the macular carotenoid composition, which can include additional nutrients such as for example but not limited to fatty acids, and at least one food grade excipient selected from the group of, but not limited to, an antioxidant, oil, a surfactant, a solubilize, an emulsifier, and the like, or a combination(s) thereof.

In one embodiment, macular carotenoid compositions and methods of treatment using the composition, may comprise one or more carotenoids in various ratios, where the carotenoid(s) are lutein and zeaxanthin, either alone or in combination, and selected from the group of, but not limited to free lutein, including lutein esters and/or isomers of lutein, zeaxanthin including one or more of its isomers, such as (R,R) zeaxanthin and/or meso-zeaxanthin, derivatives thereof.

In an embodiment, a macular carotenoid composition herein is administered in a daily dose of at or about 0.1 to 100 mg/kg body weight.

In an embodiment, a macular carotenoid composition herein is administered in a daily dose of about 1 mg to about 100 mg/kg body weight.

In some embodiments, macular carotenoid compositions herein and methods of treatment using the composition are directed to administering the composition, for example in a daily dose of about 10 mg to about 80 mg/kg body weight of macular carotenoids, to the subject in need thereof.

Further the composition may be administered such that, macular carotenoids contained therein are provided in a daily dose of about 20-50 mg/kg body weight over a certain amount of treatment to treat visual fatigue.

The methods described herein relate to treatment of eye stress disorders in subjects in need thereof, such as for example mammals including a human, who are exposed to light emitting devices of various wavelengths and intensities, for a prolonged time period, by administering an effective amount of macular carotenoid composition and evaluating one or more parameters such as effect on headache recovery, retinal photoreceptor gene expression analysis, retinal protein synthesis analysis, level of oxidative stress markers, effect on oxidative stress, effect on photoreceptor degeneration and effect on endoplasmic reticulum stress, to check beneficial effects of macular carotenoids for treatment of subjects with various eye stress disorders.

The method of preventing photoreceptor damage caused by blue light coming from digital devices requires the administration of a multicarotenoid composition. The multicarotenoid composition includes combination of lutein and zeaxanthin isomers such as meso-zeaxanthin and R,R zeaxanthin. Thus the composition is comprised of lutein, meso-zeaxanthin and R,R zeaxanthin. The macular pigments may be derived for example from a plant extract and/or oleoresin containing xanthophylls and/or xanthophylls esters, and are useful for nutrition and health applications. The macular pigments may be derived from the plant extract and/or oleoresin containing xanthophylls and/or xanthophylls esters, which is safe for human consumption. In one embodiment the ratio of lutein to zeaxanthin isomers is 5:1.

In an embodiment the isomers of zeaxanthin are present in the form of meso-zeaxanthin and RR-zeaxanthin in the ratio of 3:1. A multicarotenoid composition, for example in a test composition, may contain 1 mg trans-lutein and 0.2 mg of zeaxanthin. The zeaxanthin in this embodiment may be present as 0.15 mg of meso-zeaxanthin and 0.05 mg of R,R-zeaxanthin. The ratio of the isomers of zeaxanthin (e.g. meso-zeaxanthin and R,R-zeaxanthin) is in the range of at or about 80:20 to at or about 20:80 (e.g. 4:1 to 1:4), more preferably it is present in the range of 75:25. The composition is administered to a subject in need thereof suffering from various eye stress disorders such as intense and frequent headache, affected retinal and brain health.

In an embodiment, a test composition can be scaled to the doses described above. For example, about 0.1 mg/kg body weight of test composition (e.g. based on the amount of lutein) can include 0.1 mg of lutein and 0.02 mg of zeaxanthin isomers. In another example such as described above, about 1 mg of test composition can include 1 mg lutein and 0.2 mg zeaxanthin isomers. In another example, 50 mg of test composition can include 50 mg lutein and 10 mg zeaxanthin isomers. In another example and 100 mg test composition can include 100 mg lutein and 20 mg zeaxanthin isomers. It will be appreciated that in an embodiment, the ratio of lutein and zeaxanthin isomers is at or about 5:1 in the test composition. Accordingly about ⅕^{th} the amount of zeaxanthin isomer content is present in the composition with an amount of lutein.

In one embodiment, macular carotenoid compositions herein and methods of treatment and prevention of photoreceptor damage caused by blue light herein using the multicarotenoid compositions, may be administered to a subject in need thereof, in a form of a nutraceutical carrier, food supplement, beverage, medical food, while employing dosage forms such as granules, powders, sachets, beadlets, capsules, soft gel capsules, tablets, solutions, suspensions, and the like. The active carotenoid(s) of the compositions may be prepared by an extraction process and formulated into a composition, such as together with one or more other food grade excipient(s) and/or materials to obtain the desired form.

In one embodiment, methods of treatment/prevention of photoreceptor damage herein may comprise administering an effective amount of macular carotenoids to subjects in need thereof, to treat eye stress disorders such as intense and frequent headache, visual stress due to photoreceptor degeneration, oxidative stress and endoplasmic reticulum stress, occurring as a result from exposure to blue light, sun rays, and/or lights of varying wavelengths and/or intensities.

In one embodiment, methods of treatment/prevention of photoreceptor damage herein may comprise administering an effective amount of macular carotenoids to subjects in need thereof for treatment of eye stress disorder such as headache, and evaluating one or more parameters, such as effect on visual strain, reduction in photoreceptor degeneration, markers of endoplasmic reticulum stress, protein expressions for stress, oxidative stress markers, antioxidant enzymes and checking the effect(s) on improvement of retinal health.

Compositions and methods herein can treat various eye stress disorders, photoreceptor damage caused by blue light when symptoms are evident in subjects exposed to light sources of varying wavelengths and intensities, selected from the group of, but not limited to, electronic devices emitting blue light, UV light, flickering light, traffic signals, electronic equipment, and the like, and/or combinations thereof. More particularly the methods relates to prevention of photoreceptor damage caused by blue light exposure coming from digital devices like television sets, computers, laptops, smart phones, tablets, electronic devices, fluorescent lighting and LED lighting.

Compositions and methods herein can treat eye stress disorders, when subjects exposed for a prolonged period of time to light sources of varying wavelengths and intensities are suffering from intense and frequent headache, visual discomfort due to oxidative stress and photoreceptor degeneration and increased eye strain.

In an embodiment, subjects in need of treatment of eye stress disorders are identified and administered with an effective amount of a macular carotenoid composition, such that a daily dose of lutein alone, or zeaxanthin alone, or isomers thereof, or combinations thereof is provided to improve retinal health.

In some embodiments, methods for treatment of eye stress disorders herein and the compositions used herein, are comprised of evaluating one or more retinal health parameters such as gene expression analysis and effect on antioxidant markers.

Methods and compositions as used herein are also evaluated for in animal models (mouse) by intraperitoneal injection and eyes/retinas are then harvested for future anatomical and protein studies. Effect of compositions is checked on photoreceptor degeneration by Western blot and immunofluorescent staining on a major marker of endoplasmic reticulum stress marker. The effect of composition is also checked in animal model to check efficiency on Photo-Oxidative Retinal Damage. Effect is checked on antioxidant enzymes, photoreceptor damage caused by blue light, vision and brain health markers.

The method of preventing photoreceptor damage caused by blue light is evaluated in rat models by administration of daily oral dose of lutein zeaxanthin composition at a dose of 100 mg/kg body weight into 3 groups each exposed to different light conditions i.e. 24 hour LED light exposure, 16 hour LED light and 6 hour dark adaptation and 12 hour LED light exposure and 12 hour dark adaptation and each having treatment group and control vehicle group for a period of 60 months. At the end of 60 days the animals were sacrificed, eyes were excised and blood collected. The vision health parameters such as retinal photoreceptor gene expression retinal protein levels and levels of oxidative stress markers were measured. The gene expression of photoreceptor genes like Rhodopsin (Rho) gene, protein transcription factors nuclear factor (erythroid-derived 2)-like 2 (Nrf2),G protein subunit alpha transducin 1 gene (GNAT-1), nuclear factor-kappa B (NF-κB) glial fibrillary acid protein (GFAP), nuclear factor (NF)-κB, neural cell adhesion molecule (NCAM) gene, growth-associated protein-43 (GAP-43) gene, haemeoxygenase 1 protein expression were determined. While the compositions and methods herein have been described in terms of specific illustrative embodiments, any modifications and equivalents that would be apparent to those skilled in the art are intended to be included within the scope of the methods and compositions herein. The details of the methods and compositions herein, its objects, and advantages are explained hereunder in greater detail in relation to non-limiting exemplary illustrations.

### EXAMPLE 1

Male wistar rats (age: 8 weeks, weight: 180±20 g) were housed in a controlled environment with a 12:12-h light-dark cycle at 22° C. and provided with rat chow and water ad libitum. All experiments were conducted under the National Institutes of Health's Guidelines for the Care and Use of Laboratory Animals and approved by the Ethics Committee of the Inonu University. Rats were randomly divided into groups each containing 7 animals exposed to different light conditions based on experimental design as follows:

TABLE 1
Details of the experimental design

| | | Vehicle control |
|---|---|---|
| Groups based on light exposure | Test composition group (A) | group (B) |
| Group I - Intense LED light for a 24 hour light | Lutein zeaxanthin at a dose of 100 mg/kg | Vehicle control for |
| cycle (24 h L) | body weight for a period of 60 | a period of 60 days. |
| | days. | |
| Group II - Intense LED light for a 16 hour light | Lutein zeaxanthin at a dose of 100 mg/kg | Vehicle control for |
| and 6 hour dark adaptation(16 h L/6 h D) | body weight for a period of 60 | a period of 60 days. |
| | days. | |
| Group III - Intense LED light for a 12 hour light | Lutein zeaxanthin at a dose of 100 mg/kg | Vehicle control for |
| and 12 hour dark cycle (Light/Dark; 12 h L/12 h | body weight for a period of 60 | a period of 60 days. |
| D) | days. | |

The test composition was administered in the form of oil suspension and vehicle control was only oil, without addition of test composition in it.

At the end of 60 days animals were sacrificed at the same time each day immediately following light and dark exposure. Eyes were excised and blood collected by cardiac puncture under dim red illumination. The vision health parameters such as retinal photoreceptor gene expression retinal protein levels and levels of oxidative stress markers were measured. The gene expression of Rhodopsin (Rho) gene, protein transcription factors nuclear factor (erythroid-derived 2)-like 2 (Nrf2), G protein subunit alpha transducin 1 gene (GNAT-1), nuclear factor-kappa B (NF-κB) glial fibrillary acid protein (GFAP), nuclear factor (NF)-κB, neural cell adhesion molecule (NCAM), growth-associated protein-43 gene (GAP-43), Haemeoxygenase 1 protein expression were determined. The data was analyzed using the GLM procedure of SAS (SAS Institute: SAS User's Guide). The treatments were compared using ANOVA and student's unpaired t test; P<0.05 was considered statistically significant.

The result for retinal gene expression analysis and retinal protein synthesis are as follows:

TABLE 2
Details of the composition administered on retinal
gene expression and retinal protein synthesis

| | Intense Lights (LED) Light/Dark | | | | | |
|---|---|---|---|---|---|---|
| | 24 h L | | 16 h L/6 h D | | 12 h L/12 h D | |
| | | Test | | Test | | Test |
| Parameters | Vehicle | Compo- | Vehicle | Compo- | Vehicle | Compo- |
| tested | Control | sition | Control | sition | Control | sition |
| GNAT1 | 0.17 | 0.54 | 0.41 | 0.69 | 1.00 | 1.48 |
| NCAM | 0.37 | 0.62 | 0.64 | 0.81 | 1.00 | 1.22 |
| Rhodopsin | 0.17 | 0.58 | 0.56 | 0.78 | 1.00 | 1.63 |
| (Rho) | | | | | | |
| Rod arrestin | 0.21 | 0.61 | 0.55 | 0.69 | 1.00 | 1.42 |
| (Sag) | | | | | | |
| NFkB | 2.06 | 1.43 | 1.32 | 1.03 | 1.00 | 0.82 |
| GAP43 | 0.31 | 0.57 | 0.65 | 0.79 | 1.00 | 1.17 |
| GFAP | 1.85 | 1.26 | 1.42 | 1.09 | 1.00 | 0.81 |
| Nrf2 | 0.21 | 0.56 | 0.54 | 0.78 | 1.00 | 1.31 |
| HO-1 | 0.28 | 0.60 | 0.60 | 0.81 | 1.00 | 1.32 |

The results displayed a decreased level of retinal gene expression and retinal proteins synthesis like rhodopsin (Rho) gene, Rod arrestin (Sag)gene , GNAT-1 gene, NCAM gene, GAP-43 gene, proteins Nrf2 and HO-1 in retinal tissues and increased level of markers of oxidative stress nuclear factor kappa B(NFkB) and glial fibrillary acidic protein (GFAP) in the vehicle control group B of all the 3 groups due to damaging exposure of rat retinas to visible blue light and no treatment intervention. The treatment with test composition at a dose of 100 mg/kg body weight alleviated these effects and showed increase in the levels of gene expression and protein synthesis for Gnat1 gene, Rhodopsin gene, NCAM gene, Rod arrestin (Sag) gene, Growth associated proteins gene (GAP-43), proteins Nrf2 and Ho-1 and decrease in the oxidative stress markers GFAP, NFkB in all the three treatment groups A receiving test composition at the dose of 100 mg/kg body weight dose. As can be seen from the table the treatment groups for all the three light conditions group IA, IIA and IIIA showed an increase in expression of retinal photoreceptor gene proteins, i.e. NCAM and GNAT-1 as compared to the vehicle control.

The group I A (24 hour light group) showed almost 3 fold increase in the gene expression of GNAT-1 from 0.17 to 0.54 and a two fold increase in the level of NCAM gene from 0.37 to 0.62 as compared to the control vehicle group. Similarly the group IIIA indicated a 1.48 fold increase in the GNAT level and a 1.22 fold increase in the level of NCAM gene as compared to the control group illustrating the beneficial effect of the method of prevention of photoreceptor damage caused by blue light by administration of the multicarotenoid composition. The graphical analysis of the results is as shown in the FIGS. 1 to 9.

1. Method of preventing photoreceptor damage caused by blue light by oral administration of effective amount of macular carotenoid composition, comprising lutein, zeaxanthin and meso-zeaxanthin, through up-regulation of GNAT1 and NCAM.

2. Method of preventing photoreceptor damage of claim 1, wherein GNAT 1, a photoreceptor gene responsible for biosynthesis of alpha-transducin 1, is upregulated about 1.2 to 3.5 times in subjects administered with macular carotenoid composition as compared to subjects on the vehicle control group.

3. Method of preventing photoreceptor damage of claim 1, wherein NCAM a gene responsible for neurite growth is upregulated about 1.2 to 2.0 times in subjects administered with macular carotenoid composition as compared to subjects on the vehicle control group.

4. Method of preventing photoreceptor damage of claim 1, wherein blue light comes from the digital devices like television sets, computers, laptops, smart phones, tablets, electronic devices, fluorescent lighting and LED lighting.

5. Method of preventing photoreceptor damage of claim 1, wherein the macular carotenoid composition is comprised of lutein and zeaxanthin isomers such as meso-zeaxanthin and R,R zeaxanthin.

6. Method of preventing photoreceptor damage of claim 5, wherein effective amount of the macular carotenoid composition comprises of lutein, meso-zeaxanthin and R,R zeaxanthin ranges from 1 mg to 100 mg/kg body weight.

7. Method of preventing photoreceptor damage as claimed in claim 5, wherein the lutein and zeaxanthin isomers are present in the ratio of 5:1
